(19) United States
(12) Patent Application Publication
(10) Pub. No.: US 2025/0295391 A1
(43) Pub. Date: Sep. 25, 2025

(54) MACHINE LEARNING METHODS AND SYSTEMS TO DETECT INTERNAL MARKERS USING ULTRASOUND IMAGING

(71) Applicant: The Texas A&M University System, College Station, TX (US)

(72) Inventors: Adithyaa Karthikeyan, Bryan, TX (US); Akash Tiwari Jag Prasad, College Station, TX (US); Yuandong Wang, College Station, TX (US); Yuhao Zhong, College Station, TX (US); Satish TS Bukkapatnam, College Station, TX (US)

(21) Appl. No.: 18/638,104

(22) Filed: Apr. 17, 2024

Related U.S. Application Data
(60) Provisional application No. 63/459,742, filed on Apr. 17, 2023.

(51) Int. Cl. A61B 8/00; G06N 3/0464; G16H 30/40

(52) U.S. Cl. CPC A61B 8/5207; G06N 3/0464; G16H 30/40

(57) ABSTRACT

Systems, methods, and computer readable media for detecting internal markers using ultrasound imaging. In some examples, a method includes using ultrasound imaging to scan an interior of a target object. The method includes providing scanned images generated by the ultrasound imaging as input to a machine learning software system configured to host a convolutional neural network (CNN) model. The method includes detecting, by the CNN model, a location of at least one internal marker inside the target object using the scanned images. The method includes extracting identification information stored in the at least one marker.

### PRIORITY CLAIM

This application claims the benefit of U.S. Provisional Patent Application Ser. No. 63/459,742, filed Apr. 17, 2023, the disclosure of which is incorporated herein by reference in its entirety.

### GRANT STATEMENT

This invention was made with government support under Grant Nos. 1849085 and 1953694 awarded by the National Science Foundation. The government has certain rights in the invention.

### BACKGROUND

Although the manufacturing community has been at the forefront of adopting the sensor and Al technologies for industrial quality assurance, inspection of bulk products to detect internal morphological defects remains slow, expensive, and imprecise, even with the latest non-destructive evaluation (NDE) techniques. For example, X-ray computed tomography typically takes 5-15 hours to scan products as small as 100 mm³ in volume. Other NDE techniques, such as conventional eddy current, thermography, ferromagnetic, and chemical profiling suffer from limited (a few mm) depth of penetration and poor detection-sensitivity for various materials. Consequently, even the quality-critical microelectronics industry bypasses bulk product inspection, and instead it mostly tests the functional performance.

### SUMMARY

This document describes systems, methods, and computer readable media for detecting internal markers using ultrasound imaging. In some examples, a method includes using ultrasound imaging to scan an interior of a target object. The method includes providing scanned images generated by the ultrasound imaging as input to a machine learning software system configured to host a convolutional neural network (CNN) model. The method includes detecting, by the CNN model, a location of at least one internal marker inside the target object using the scanned images. The method includes extracting identification information stored in the at least one marker.

The computer systems described herein can be implemented in software in combination with hardware and/or firmware. For example, the subject matter described herein can be implemented in software executed by a processor. In one example implementation, the subject matter described herein may be implemented using at least one computer readable medium having stored thereon computer executable instructions that when executed by the processor of a computer cause the computer to perform steps or operations. Exemplary computer readable media suitable for implementing the subject matter described herein include non-transitory devices, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

FIGS. 1A-1C illustrate the operating principle of ultrasound imaging. FIG. 1A shows acoustic mismatch (Z₁-Z₂) at a boundary B. FIG. 1B sows the signal received at piezoelectric element 1. FIG. 1C shows a representative ultrasound image.

FIG. 2 shows an illustration of the LIME scheme.

FIGS. 3A-3B shows an example test product. FIG. 3A shows isometric, front, and right-side views of the 2 cm cubic test product embedded with a distribution of spherical markers. FIG. 3B shows a 3D printed cube made of Veroclear material.

FIG. 4A shows a schematic of the ultrasound scanning experimental setup. FIG. 4B shows a representation of the product scanning showing image frames and different regions of marker distribution, and the marker coordinates.

FIGS. 5A-5B show a box plot summary of the prediction accuracies for training, validation, and testing datasets over the 10-fold cross validation models corresponding to (FIG. 5A) CNN1 for the detection of markers at shallow depths (FIG. 5B) CNN2 for the detection of markers at greater depths.

FIGS. 6A-6F show example view of the cube. FIG. 6a shows a representative view of the cube's actual design on its top section in region A. FIG. 6B shows an ultrasound image of a frame captured in region A. FIG. 6C shows the corresponding LIME explanation highlighting the important segments that determine the presence of artifacts. FIG. 6D is side view of the cube's design in region B. FIG. 6E is an ultrasound image of a frame captured in region B. FIG. 6F shows the corresponding LIME interpretation highlighting the important segments that determine the absence of artifacts.

FIG. 7A shows an ultrasound image of a correctly classified frame captured in region C of the scan containing markers at a depth of 9-12 mm near the centre of the cube. FIG. 7B shows the corresponding LIME explanation highlighting the important segments that indicate that the frame is from region C.

FIG. 8 is a block diagram of an example system for ultrasound imaging.

### DETAILED DESCRIPTION

The technology contains an ultrasound reading device and/or transducer and a machine learning software. The ultrasound transducer scans the interior of 3D printed parts for markers. In some embodiments, the disclosed subject matter specifically focuses on 3D printed parts made of Veroclear polymer. The scanning images obtained from the transducer is processed by a machine learning software to identify markers in these images. In this manner, the information stored in these markers is extracted based on their physical locations within the 3D printed part.

At present, anticounterfeit tags introduced on the surface of physical products can be tampered, thus destroying the ability to identify these parts. Introducing these anticounterfeit tags inside parts can improve the integrity of these tags. 3D printing technologies enable the introduction of tags inside the parts, but presently, no technology can achieve “quick” and “accurate” scans that will extract information stored in these parts. Extant technologies lack in either one of these two capabilities. The disclosed technology uses fast scanning techniques—specifically ultrasound imaging—coupled with machine learning software—specifically a convolutional deep learning framework—to obtain speed and accuracy in scanning the internal markers present inside 3D printed parts.

The unique feature of the disclosed technology involves the application of a machine learning framework to detect location of markers inside 3D printed parts as captured by ultrasound images. Based on recent developments in the field of explainable machine learning, we are able to assess the trust in the machine learning model predictions to accurately detect the presence of markers and their locations. Ultrasound scanning by itself, although fast, can be limited in image resolution based on the material type being scanned. The introduction of the machine learning framework allows us to work with the limited resolution to accurately detect the marker locations.

Moreover, CT scanning technology is currently being researched to obtain internal scans of parts with markers. However CT scans can be very time consuming for being deployed in a mass production environment. Surface marker technologies are widely used to identify parts. These include QR codes, bar codes, and the like, but their presence on the surface makes them vulnerable to damage. Alternate identification strategy includes RFID tags, but such tags can be expensive and cannot be introduced on all types of manufactured parts.

The disclosed technology relies on introducing markers inside of physical 3D printed parts, which can be made less intrusive to the functionalities of the parts by varying the size and location of the markers unlike RFID tags. Markers are present inside of 3D printed parts and are therefore hard to tamper with. Ultrasound devices coupled with machine learning software can improve scanning time and accuracy which can be acceptable in a mass production environment.

CT scanning for a 2 cm cube can take up to 8 hours while scanning with ultrasound devices can take 30 seconds. Ultrasound scanning technology can obtain phenomenal improvements in scanning times but can have poor resolution in comparison to CT scans. However, to read the information stored in markers, we are only required to read the location of the embedded markers accurately without any concern on the dimensional accuracy. With a low resolution scan, a machine learning framework can allow for the extraction of the location of the markers with acceptable accuracy. In some embodiments, the LIME framework from the field of explainable machine learning is used to identify marker locations based on the development of a deep learning classifier.

Further details of the presently disclosed subject matter can be found in the following discussion which describes examples and test systems for explainable AI—infused ultrasonic inspection for internal defect detection with reference to studies performed.

### Introduction

Although the manufacturing community has been at the forefront of adopting the sensor and Al technologies for industrial quality assurance, inspection of bulk products to detect internal morphological defects remains slow, expensive, and imprecise, even with the latest non-destructive evaluation (NDE) techniques. For example, X-ray computed tomography typically takes 5-15 hours to scan products as small as 100 mm³ in volume. Other NDE techniques, such as conventional eddy current, thermography, ferromagnetic, and chemical profiling suffer from limited (a few mm) depth of penetration and poor detection-sensitivity for various material. Consequently, even the quality-critical microelectronics industry bypasses bulk product inspection, and instead it mostly tests the functional performance.

The limitations of the current bulk inspection technologies also impede the assurance of authenticity of products against counterfeiting and cyber-attacks. Reflecting the emergence of a manufacturing-as-a-service (MaaS) paradigm, industrial product authentication technologies are growing at an annual rate of over 10% to exceed $250B by 2026. The prominent technologies, employing QR codes, radiofrequency identification, etc., merely mark the product packaging or at best, a product surface. These surface markers are vulnerable to tamper and wear. Attempts have been made to embed alternative geometric (e.g., 3D QR codes) and intrinsic markers (e.g., morphological or microstructure features). As with quality assurance, the limitations of the current bulk product inspection curtail the viability of these innovative authentication methods.

Interestingly, the rapidly increasing resolutions and cost reductions make imaging technologies attractive for bulk inspection to assure both the quality and authenticity of products. While the manufactured parts are opaque to optical imaging, reverberation and attenuation effects severely degrade the signals from various electromagnetic imaging methods, leading to poor quality and authenticity assurance. Ultrasound methods are considered the most promising for bulk inspection of manufactured products.

Ultrasound imaging instruments use piezoelectric elements to release ultrasound pressure waves into a product at frequencies of up to 100 MHz. The boundaries of various internal artifacts (e.g., defects and authentication markers) of the product generate echoes of incident ultrasound. Images of the internal structure of the product are reconstructed based on the intensity of echo received from varying depths of the product. Ultrasound imaging is quick and can be performed in real time with 25-100 fps rate, in contrast to other inspection techniques.

Although fast and cost-effective, the sensitivity of ultrasound sensors tends to be poor, especially to discern the artifacts (e.g., defects and embedded codes) in many polymeric and composite materials. Infusing the physics-based image reconstruction with recent AI methods can enhance the detection of the product artifacts one leap forward. Among the AI methods, several supervised machine learning methods have been applied to enhance the sensitivity and the prediction power of NDE and ultrasound methods. They predominantly used experimental data to learn purely “black box” models to segment or classify the artifacts in ultrasound images. The models are neither based on nor contribute to the understanding of the underlying physical phenomena. Hence, they cannot be generalized beyond the training scenarios.

In contrast, the new class of explainable AI (XAI) approaches would not just complement the predictions from powerful machine learning methods, but would help discover the underlying physical processes. Also, different from prior applications, this work aims to employ machine learning methods to enhance the discrimination power of the ultrasound images beyond what purely physics-driven image renderings would achieve.

Recent results in an XAI method called local interpretable model-agnostic explanations (LIME) are adapted to discover the physical relationships captured in these machine learning models. This novel approach is applied to discern, using ultrasound imaging, the markers (artifacts) distributed in polymeric blocks to mimic pores and authentication codes. Even the state-of-the-art ultrasound image segmentation methods could not detect these markers. The results suggest that the present approach can detect these markers to accuracies exceeding 80%. More importantly, this approach provides a robust, fast, and cost-effective solution to the quality and cybersecurity challenges in the emerging MaaS paradigm.

### XAI-Infused Ultrasound Inspection

### Principles of Ultrasound Inspection

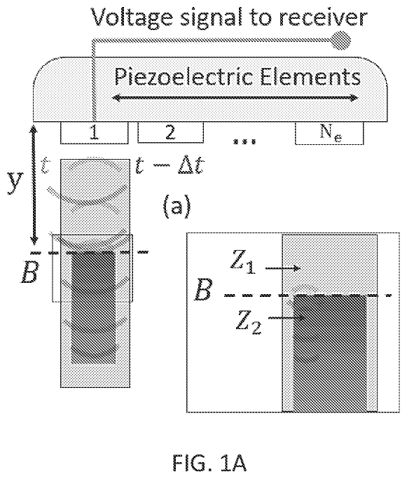

FIG.1 illustrates operating schematics of ultrasound imaging. It employs multiple piezoelectric elements within a transducer that generate and receive pressure waves.

Pressure waves generated from an element travel downward into the product. Their propagation velocity c(y) at depth y is determined by acoustic impedance Z(y) and density p(y) of the material at location y of the product as c(y)=Z(y)/p(y). Whenever the pressure waves encounter an artifact, causing a major inhomogeneity in media (FIG. 1A), the waves are scattered at boundaries B within the product. A portion of the incident wave is reflected back to the piezoelectric element. Intensity of the reflected wave Iᵣ in relation to incident wave Iᵢ depends on the acoustic impendences Z₁ and Z₂ of the media on both sides of the boundary B as

$${I}_{r} / {I}_{i} = {( {Z}_{1} - {Z}_{2} )}^{2} / {( {Z}_{1} + {Z}_{2} )}^{2} \qquad ( 1 )$$

The piezoelectric elements generate a voltage signal vᵣ (t) at time t by aggregating the reflected wave (echo) it receives as

$${v}_{r} ( t ) = {v}_{p e} ( t ) * ( {f}_{m} ( y ) * {h}_{p e} ( y , t ) ) \qquad ( 2 )$$

where ‘*’ denotes a convolution sum (aggregation), fₘ(y)=(p(y)−p₀)/p₀−2(c(y)−c₀)/c₀ is the quantity of interest to capture in the ultrasound image and it captures inhomogeneity within the object, Po and co are the nominal values of the density and velocity, respectively, vₚₑ(t) is the excitation impulse response that aggregates fₘ(y) in time, hₚₑ(y,t) is the spatial impulse response of the pulse echo relating the transducer geometry to the acoustic field that aggregates fₘ(y) in space.

The ultrasound image is in effect obtained by inverting Eq. (2) to retrieve a surrogate of fₘ(y), and this results in a single image line from the crystal element (FIG. 1B). The line reconstructions from vᵣ(t) obtained from multiple piezoelectric elements are juxtaposed to obtain the signal s. The final rendering of the ultrasound image is given by ξ=Ψ{G [V{R₀(s)}]}, where Ro is a reconstruction operator (that performs the inversion operation outlined earlier), V{·} is a nonlinear operator that determines the intensities, G is used to achieve the correct image dimension and Ψ{·} is an optional operator for image enhancement and noise filtering.

In reality, the quality of the image ξ depends largely on the acoustic mismatch Z₁-Z₂ at boundary B. A small acoustic mismatch results in poor contrast due to a very small echo generated at this depth. If the impendence of the material is high (as with polymers), the propagating waves, and hence the signals are further attenuated. Also, large acoustic mismatches degrade the resolution of the images below this depth. Additionally, side lobes are produced in the ultrasound beam causing the signals to travel in alternate directions and positioning errors. These phenomena and their manifestation in the ultrasound images for various material systems are not well understood. Recent advances in XAI can help gain a deeper understanding of how these complex phenomena ultrasound manifest in the ultrasound images.

### XAI and Local Interpretable Model-Agnostic Explanation (LIME)

Despite decades of application of the ultrasound techniques, their physical understandings are not mature yet to correctly predict the various spatial patterns in the reconstructed ultrasound images, even for simple realistic products. The growing suite of sophisticated machine learning methods, such as deep convolution neural networks (CNNs) trained with labelled experimental ultrasound images can detect the internal defects and markers in manufactured products at orders of magnitude higher resolutions than what is possible with the current ultrasound reconstruction methods. However, the high predictive power of these advanced machine learning methods comes at the expense of poor interpretability. While these methods can accurately detect the presence of artifacts, it is not straightforward to locate them. Explainable AI (XAI) and local Interpretation model-agnostic explanation (LIME) approaches are garnering significant interest to explain the complex “black box” machine learning models. They can be adapted to further analyse black box models to locate the internal artifacts of a product from an ultrasound image, as well as to discover the dominant physical mechanisms that cause certain unusual ultrasound features to appear whenever a specific internal artifact is present.

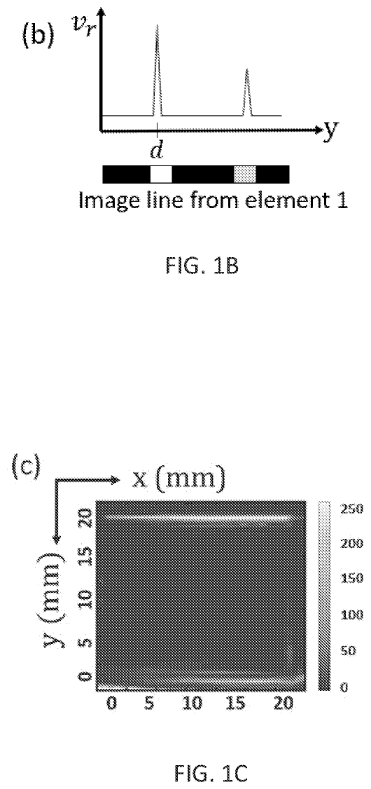

Instead of considering the complex functional relationship that CNN and other black box models capture, LIME aims to explain the relationship local to the various neighbourhoods of the input space (here, a neighbourhood consists of ultrasound images that are similar to each other). This is achieved by generating synthetic perturbed image samples μᵢ (see FIG. 2) within the neighbourhood of an input ξ and constructing a highly interpretable linear model using these samples that will hold only for that neighbourhood.

The coefficients β of the resulting linear model can suggest the importance of the various components (here, a group of pixels) of an input (i.e., an imageξ). To explain the prediction of an already trained CNN classifier g aroundξ, LIME first partitions ξ into M segments. Then, a binary vector ξ′ ∈{1} is used to alternatively represent the original imageξ, where each element of ξ′ indicates the presence of a segment of ξ. By randomly setting some elements in ξ′ to 0, LIME further generates n different binary vectors, μᵢ^{′} ∈{0,1}^{M}, i =1,2, . . . , n. Each of these generated vectors represents a perturbed image ui (i =1, 2, . . . , n) of ξ. Each perturbed image ut is weighted by a similarity index π_{ξ}(μᵢ) as

$${π}_{ξ} ( {μ}_{i} ) = exp ( - {D ( {ξ}^{′} , {μ}_{i}^{′} )}^{2} / {δ}^{2} ) , \qquad ( 3 )$$

where δ is the kernel width. The distance D (ξ′, μ_{i′}) is the cosine of the angle between ξ′ and μᵢ^{′}. Next, using μᵢ^{′} as inputs and the corresponding outputs from g as the ground truth, LIME trains a linear model, defined by its coefficient vector β* as:

$${β}^{*} = {arg min}_{β} {\sum }_{i = 0}^{n} {{π}_{ξ} ( {μ}_{i} ) [ g ( {μ}_{i} ) - 〈 β , {μ}_{i}^{′} 〉 ]}^{2} + λ 〈 β , β 〉 , \qquad ( 4 )$$

where μ₀=ξ, μₒ^{′}=ξ′, g (μᵢ) is the output of the CNN model, and λ is the ridge regularization term. The sign and magnitude of each element of β* indicate positive/negative importance of the presence of the corresponding segment in ξ. Thus, a segment with a positive element implies that without the segment the image is less likely to be classified to have markers, and a negative coefficient implies the opposite.

Additionally, a total of 2^{M}−1 of possible perturbations exist to estimate β* about every image ξ. The number of segments M is typically of the order of 10², and employing all possible permutations is computationally intractable. Therefore, a much smaller perturbation sample size n is usually chosen. This sampling introduces uncertainty in the resulting feature importance β*. The following theoretical result was used to guide the selection of n.

Theorem 1. (Perturbed sample size) [18]: The number of perturbed samples n required to achieve an uncertainty interval width w of feature importance at a user-specified confidence level α can be calculated as

$$n = 4 {ε}_{J}^{2} / \{ {\overline{π}}_{J} \times {[ w / {Φ}^{- 1} ( α ) ]}^{2} \} , \qquad ( 5 )$$

where π̅_{J}=Σᵢ₌ₒ^{J}π_{ξ}(μᵢ)/J is the average weight for the perturbations estimated from an initial J samples, ϵ_{J}² is the empirical sum of squared errors between the LIME linear model and g, weighted by π_{ξ}(μᵢ) for i=1, . . . , J, and φ⁻¹(α) is the two-tailed inverse normal cumulative distribution function at confidence level α.

It can be observed that, to keep the uncertainty interval w small, n needs to be large. On the other hand, when the error ϵ_{J}² is large, i.e., when the LIME model cannot accurately approximate g locally and thus cannot provide high-quality explanations, we also need a proportionally larger number of perturbed sample size. Therefore, it is recommended to only consider the explanations obtained from the LIME models that have a sufficient accuracy. Here, criteria such as R² (coefficient of determination) can be used to evaluate the performance of the LIME model.

### Case Study and Results

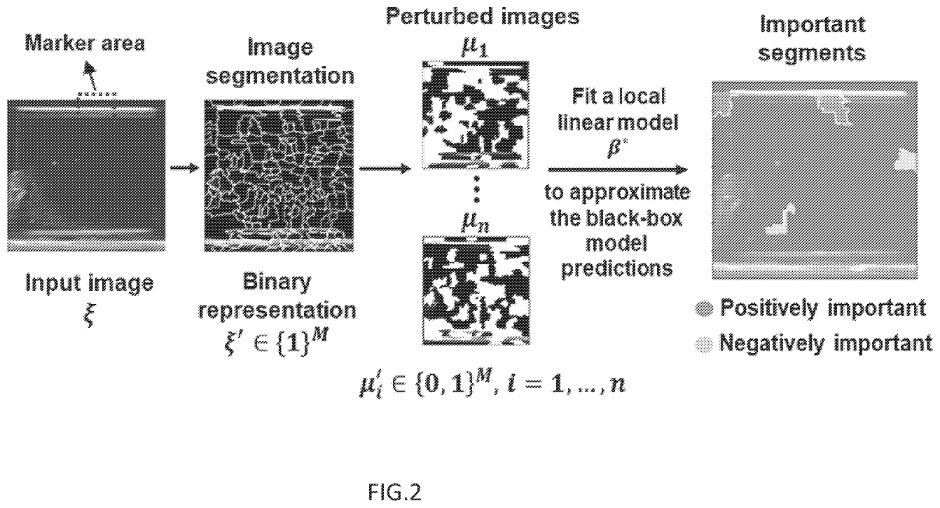

To assess the performance of the XAl-infused ultrasound inspection, we considered 2 cm cubic test products made of a transparent polymer (Veroclear). The products are embedded with spherical markers of diameter 0.48 mm (see FIG. 3 (a)). The markers are dispersed at both shallow (1-3 mm depth) and deeper (9-12 mm depth) locations, emulating the occurrence of defects (e.g., internal pores and voids) and embedded authentication codes in a product. The products were printed using a Stratasys J750 Polyjet printer and inspected using an ultrasound imaging system from Aixplorer Ultimate with an SL 15-4 transducer. Although the difference in the acoustic impedance between Veroclear (˜3×10⁶ Pa s m-⁻¹) and air (415 Pas m⁻¹) assures sufficient contrast to identify the markers in the ultrasound images, the large acoustic attenuation coefficient of Veroclear (˜170 dB m⁻¹ compared to <10 dB m⁻¹ for metallic materials at 2 MHz) severely impedes the discernibility of markers located more than 5 mm below a surface.

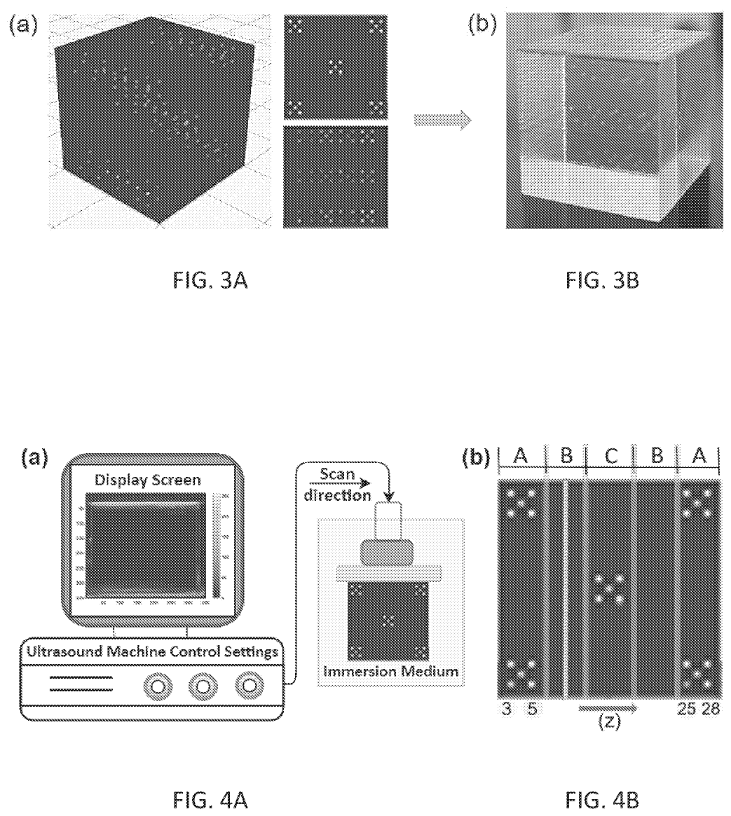

The experiments consisted of moving the components at a speed of ˜1 mm/sec relative to the ultrasound instrument to collect image-frames at 18 different settings of frequency (5, 7.5, and 12 MHZ), input power (OdB, −5 dB and −10 dB) and focal position (Top and bottom) (FIG. 4A). Each image frame, shown as a light blue cross-section plane FIG. 4b, captures the markers located underneath the instrument at a specified time during a scan. The numbers indicated at the bottom of FIG. 4C represents the time (in seconds) at which the instrument traverses the cross section. Also, the higher the frequency setting, the higher are the resolution of the scan and signal attenuation. The higher the power, the greater is the scan illuminance (and scattering effects). The focal position indicates the depth that requires the best resolution. Also, based on the maker patterns, the ultrasound image-frames can be generally labelled into three classes, namely, those that capture the shallow markers (Region A), deeper markers (Region C) and no markers (Region B). The markers are distributed over the depths of 0.5-3 mm from the top section for Region A, and 9-12 mm for Region C.

To apply the novel XAI-infused ultrasound method, CNN models are employed to classify these ultrasound image frames depending on whether a frame contains embedded markers. A CNN model extracts a compact set of features from an image via convolution. Then, it associates these features with the prespecified class of the image. In this way, it learns the feature pattern of each class and can classify a new image into the most likely class. A good classification accuracy may indicate that the CNN has learned to distinguish the ultrasound image frames that contain the markers.

One of the challenges encountered here is that the presence of markers is visible, even partially, only up to a depth of 3 mm from the top of the cube. The markers embedded at depths greater than 3 mm from the top of the cube such as those present in Region C and bottom sections of Region A, are not observable even with advanced image segmentation methods. However, the presence of markers can modify the ultrasound image intensity patterns at locations far away from where the markers are placed. These correlated patterns across the image, caused by the presence of the marker, serve as a signature that can be learned by a CNN model.

FIG. 3A shows isometric, front, and right-side views of the 2 cm cubic test product embedded with a distribution of spherical markers. FIG. 3B shows a 3D printed cube made of Veroclear material.

In this study, two different CNN models are considered: CNN1 to detect the shallow markers, and CNN2 to detect the markers at greater depths. The models take an image-frame as input and outputs 0 or 1 indicating the absence or presence of markers. The architecture of both CNNs comprises of three convolution layers having 32, 64 and 128 filters, respectively, followed by a max pooling layer, and finally a fully connected dense layer with 128 neurons. A stochastic gradient-descent optimizer is used to minimize loss and a binary cross entropy quantifier is used to measure the accuracy.

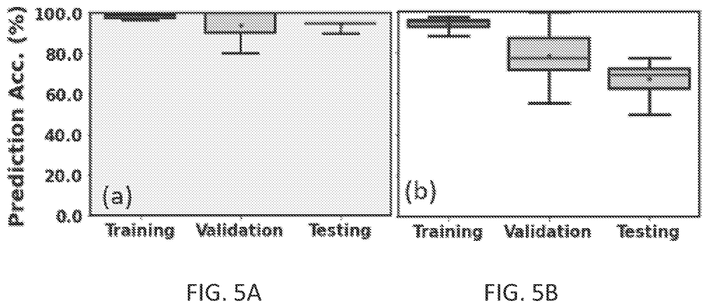

CNN1 used the dataset from Regions A and B. For this analysis, a total of 123 frames were collected, of which 69 frames were sampled from region A, and 54 frames (that are devoid of any markers) were sampled from region B of the cube (refer FIG. 4(b)). This data was then split into training and testing sets in the ratio of 0.8, and the model was trained for 50 epochs. A 10-fold cross validation on the training set was performed to mitigate bias in data splitting, and to better evaluate the model's performance over the testing dataset. The CNN1 model consistently achieved an accuracy of 94.9% on the test dataset as illustrated by the box plot in FIG. 5 (a).

CNN2 was used to detect the presence of markers at greater depths (i.e., in Region C). It used 62 frames from Region B and 50 frames from region C. This data was split into training and testing sets in the ratio 0.8 and a 10-fold cross validation was performed as before. The model can achieve a prediction accuracy of 90% on the validation set and 78% with the testing set (FIG. 5B). Given the sparsity of data there is a chance that the models might have been overtrained. However, they possess considerable explanatory power as conveyed by the LIME Analysis. The sensitivity and specificity of detecting the markers can be improved further by fusing inspections made along different directions and combining multiple redundant marker measurements recorded in the vicinity of the marker region.

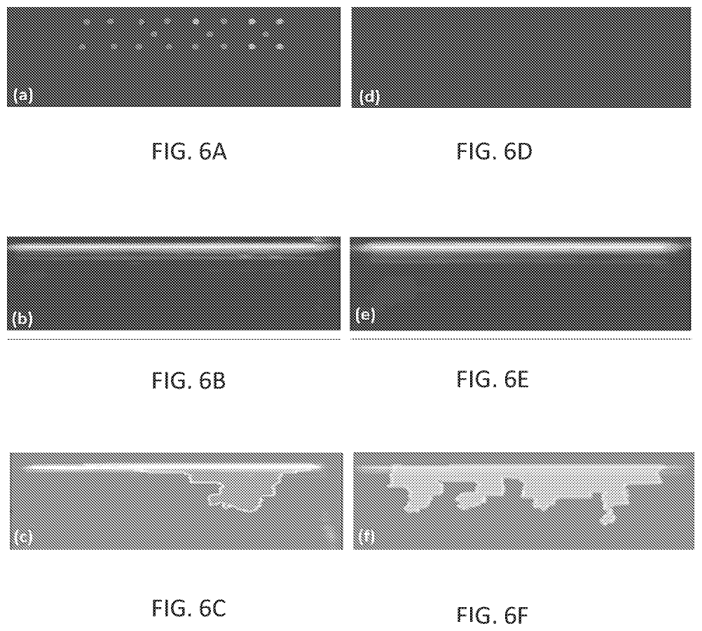

FIGS. 6A-6C summarizes a representative result from LIME analysis of CNN1. The markers present along a section (FIG. 6A) are hardly noticeable in the raw ultrasound images (FIG. 6B). The two bright spots near the top right corner of the image are the only possible indicators for the presence of markers. In contrast, the segments from the LIME analysis (FIG. 6C) strongly indicate the presence of markers in the image-frame at these locations. Similarly, FIG. 6E depicts the ultrasound scan pertaining to a frame without any markers. The green segments from LIME analysis (FIG. 6F) contribute to strongly indicating the absence of embedded markers in the frame.

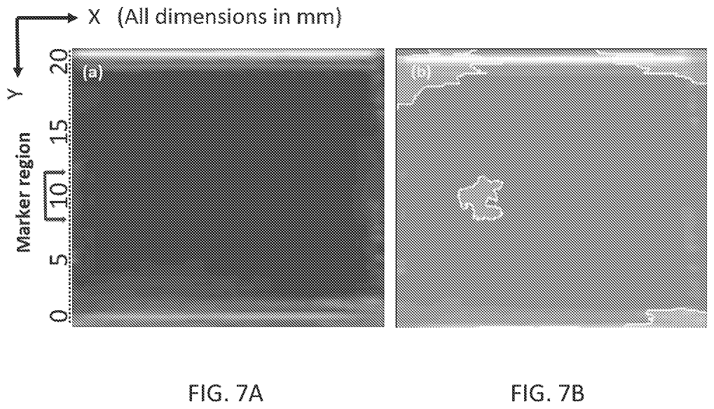

Similarly, for the more challenging case of CNN2 for detecting markers in Region C, the red segments near the middle as depicted in FIG. 7B indicate the presence of embedded markers in the image-frame. The important segments near the top corners of the frame and, interestingly, far away from marker locations, are likely formed due to the scattering and reverberations of sound waves in the presence of the deeper markers. This is somewhat like a “butterfly effect”, where one needs to look for intensity variations elsewhere to locate a defect at a particular location. Pertinently, the XAI-infused approach helps in determining where to look for these crucial patterns, and these discovered patterns-largely ignored in the ultrasound literature-help with detecting artifacts located 3-4 times deeper compared to conventional ultrasonic inspections.

### Conclusions

A novel explainable AI (XAI)-infused ultrasound imaging principle that enables a fast, holistic inspection of products manufactured from diverse materials, and detection of internal artifacts such as voids, pores and other defects is presented. Based on LIME analysis, it was discovered that distinct correlated spatial patterns are formed in the ultrasound images at locations that are far away from the artifact in the polymer cubic component with embedded markers. The results suggest that CNN models can detect internal artifacts that could not be discerned using any existing image segmentation method to accuracies exceeding 80% by effectively capturing the discovered spatial patterns. This result can profoundly impact the assurance of not just product quality, but also authentication and cybersecurity in the emerging manufacturing paradigm.

### EBLIME: Enhanced Bayesian Local Interpretable Model-Agnostic Explanations

EBLIME can be used to explain black-box machine learning models and obtain the distribution of feature importance using Bayesian ridge regression models. Compared to some conventional methods, EBLIME yields more intuitive and accurate results, with better uncertainty quantification in terms of deriving the posterior distribution, credible intervals, and rankings of the feature importance.

### Prior Assumptions

Given a perturbed dataset Z and the corresponding blackbox prediction Y, our proposed explanation model has the following hierarchical form

$$Y \vert Z , β , ϵ ∼ Z β + ϵ \qquad ( 2 a )$$

$$ϵ \vert {σ}^{2} ∼ 𝒩 ( 0 , {diag}^{- 1} ( {Π}_{x} ( Z ) ) {σ}^{2} ) \qquad ( 2 b )$$

$$β \vert {σ}^{2} , λ ∼ 𝒩 ( 0 , {λ}^{- 1} {σ}^{2} {𝕀}_{p} ) \qquad ( 2 c )$$

$${λ}^{- 1 / 2} ∼ half - Cauchy ( 0 , 1 ) \qquad ( 2 d )$$

$${σ}^{2} ∼ Inverse - Gamma ( a , b ) \qquad ( 2 e )$$

$$P ( λ , {σ}^{2} ) = P ( λ ) P ( {σ}^{2} )$$

wherein ϵ is the error of using the Bayesian ridge regression model to approximate the black-box model. Here, each element of is modeled as a zero-mean Gaussian noise with variance inversely related to the weight. In other words, the error is more uncertain for perturbed instances that are farther from the original input x.

It is important to note that in BayesLIME (Slack et al., 2021), both ϵ and β are conditioned on σ² which captures the underlying uncertainty. Their intuition is that the smaller the uncertainty of the error is, the more confident we are supposed to be about the resulting feature importance. Therefore, BayesLIME simply assumed β and ϵ to have the same covariance (the effect of the constant weight matrix in Equation (2b) can be ignored). This, however, can discount or exaggerate the uncertainty of β. To address this problem, we introduce a random variable λ>0 which effectively scales the covariance of β and works as a ridge parameter.

For σ², similar to BayesLIME, we choose a weakly informative inverse-gamma conjugate prior with parameters a and b, i.e., P(σ²)∝(σ²)^{-a-1} exp (−b/σ²). However, we use a larger value (e.g., 1) for a and b, instead of 10⁻⁶ in BayesLIME. This results in heavier-tailed prior and posterior densities that assign less mass near σ²=0, allowing larger values of σ² to be plausible.

For the ridge parameter λ, we use a default half-Cauchy prior on the scale λ^{-1/2}. By change of variables, this gives us P(λ)∝λ^{-1/2}(1+λ)⁻¹)1_{(0∞)}(λ). Such a prior has better frequentist operating characteristics compared to the commonly adopted conjugate inverse-gamma prior. It is also heavier-tailed, allowing the variability of λ to be either very small or large.

### Inference

Our goal is to obtain the marginal posterior of β, so that we can analyze the uncertainty of the feature importance given a specific perturbed dataset Z and Y.

Proposition. Using Bayesian conjugacy adapted to the weighted ridge regression setup, we can write

$$β \vert {σ}^{2} , λ , Y ∼ 𝒩 ( \hat{β} , {V}_{λ} {σ}^{2} ) \qquad ( 3 )$$

$${σ}^{2} \vert λ , Y ∼ Inverse - Gamma ( a + N / 2 , {Q}_{λ} / 2 ) \qquad ( 4 )$$

$$wherein {Q}_{λ} = {{Y}^{T} ( {diag}^{- 1} ( {Π}_{x} ( Z ) ) + {λ}^{- 1} {ZZ}^{T} )}^{- 1} Y + 2 b .$$

Based on the conditional posterior of 6, we can estimate the marginal posterior mean E (β|Y) and covariance Cov (β|Y) using Algorithm 1. The marginal posterior provides insights into the distribution of the feature importance β, instead of merely a point estimation provided by LIME. To implement Algorithm 1, we further derive the following proposition.

Proposition. The posterior density of λ can be written as

$$P ( λ \vert Y ) ∝ {\vert {M}_{λ} \vert}^{- 1 / 2} {( {Q}_{λ} )}^{- ( a + N / 2 )} P ( λ ) . \qquad ( 5 )$$

wherein M_{λ}=diag⁻¹(πₓ(Z))+λ⁻¹ZZ^{T}. The posterior mean of λ can be approximated by discretizing P(λ|Y) over {λ₁, . . . , AL}, that is

$$E ( λ \vert Y ) \approx {\sum }_{l = 1}^{L} {λ}_{l} {\vert {M}_{{λ}_{l}} \vert}^{- 1 / 2} {( {Q}_{{λ}_{l}} )}^{- ( a + N / 2 )} {P}^{′} ( {λ}_{l} ) \qquad ( 6 )$$

wherein L is the number of discretized λ values and P⁰(λ) denotes the normalized P(λ) satisfying Σₗ₌₁^{L}P′(λₗ)=1.

Algorithm 1 Estimate E(β|Y) and Cov(β|Y)

| | |
|---|---|
| 1: | Input: P(λ\|Y), P(σ²\|λ, Y), P(β\|σ², λ, Y), number |
| | of posterior samples s |
| 2: | for i = 1 to s do |
| 3: | Sample λᵢ from P(λ\|Y) |
| 4: | Sample σᵢ² from P(σ²\|λᵢ, Y) |
| 5: | Sample β₍ᵢ₎ from P(β\|σᵢ², λᵢ, Y) |
| 6: | end for |
| 7: | return E(β\|Y) and Cov(β\|Y) based on |
| | {β₍₁₎, ..., β₍ₛ₎} |

In Equation (6), MA and Qx are functions of Z. This means E(λ|Y) varies with different perturbed datasets Z, not to mention different inputs x. Therefore, existing methods that simply set λ to be a constant (i.e., β and sharing a covariance of the same scale) may produce problematic results.

Sampling λ's from P(λ|Y) can be difficult when Nis large because the magnitude of Q_{λ}can be numerically troublesome. Therefore, Gumbel trick can be employed (see Algorithm 2).

Algorithm 2 Sample λ's from P(λ|Y) using Gumbel trick

1: Input: discretization set size L, posterior sample size s
2: Initialize S = { } as the set of sampled λ.
3: Suppose P(λ|Y) is discretized over {λ₁, . . . , λ_{L}}.
4: for i = 1 to s do
5: for l = 1 to L do
6: gₗ = log P′(λₗ) − (1/2) log |Mλₗ| − (a +
N/2) log Q_{λ}_{ₗ}
7: $Draw {μ}_{l} {∼}^{i . i . d} Exponential ( 1 )$
8: Uₗ = − log μₗ
9: end for
10: l* = arg max_{l∈(1, . . . , L}} (gₗ + Uₗ)
11: S.append(λₗ*)
12: end for
13: return S

Guidance on setting the upper bound r when creating the discretization set {λ₁, . . . , λ_{L}}: based on the shape of P(λ) over λϵ (0, ∞), r can be determined such that P(r) is less than a threshold close to 0.

This section described a Bayesian ridge regression approach—EBLIME to explain black-box machine learning models, i.e., quantify features' contribution (importance) to the model's prediction on a given input as well as the underlying uncertainty. The method aims to enhance a prior Bayesian explanation method in the following way. The prior method assumes that the feature importance B and the error of the linear explanation model € share the same uncertainty. This assumption has been shown to severely impede the performance of the earlier method in certain cases. In contrast, EBLIME had aimed to improve the explanation performance by introducing a random variable, a regularization parameter λ to scale the covariance of β.

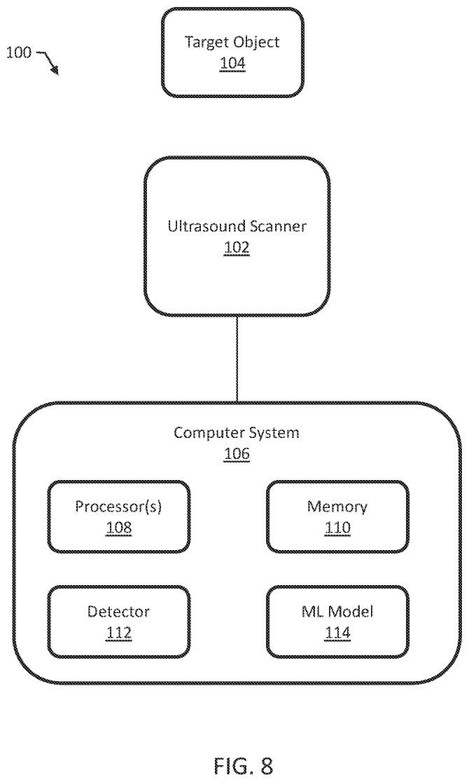

FIG. 8 is a block diagram of an example system 100 for inspection by an ultrasound scanner 102 of a target object 104. The ultrasound scanner 102 is ocnfigured to scan the interior of the target object 104. The target object 104 can be, e.g., a 3D printed part. The target object 104 can include a metallic material, a plastic mateiral, or a composite material.

The system 100 includes a computer system 106 configured to receive scanned images from the ultrasound scanner 102. The computer system 106 includes one or more processors 108 and memory 110 storing instructions for the processors 108. The computer system 106 includes a detector 112 implemented on the processors 108 and configured for hosting a machine learning model 114. The detector 112 uses the model 114 for detecting a location of at least one internal marker inside the target object using the scanned images. The detector 112 extracts identification information stored in the at least one marker.

The marker can include a code or material defect. In some cases, the code is an embedded bar code or an embedded quick response (QR) code. The code can be used to authenticate the target object 104.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method comprising:
using ultrasound imaging to scan an interior of a target object;
providing scanned images generated by the ultrasound imaging as input to a machine learning software system configured to host a convolutional neural network (CNN) model;
detecting, by the CNN model, a location of at least one internal marker inside the target object using the scanned images; and
extracting identification information stored in the at least one marker.

2. The method of claim 1 wherein the at least one marker includes at least one code or material defect.

3. The method of claim 2 wherein the at least one code is an embedded bar code or an embedded quick response (QR) code.

4. The method of claim 2 wherein the at least one code is used to authenticate the material object.

5. The method of claim 1 wherein the target object comprises a three dimensional (3D) printed part.

6. The method of claim 1 wherein the target object comprises one or more of a metallic material, a plastic material, or a composite material.

7. The method of claim 1 wherein the CNN model utilizes explainable artificial intelligence (XAI).

8. The method of claim 1 wherein the ultrasound imaging is conducted using a transducer element.

9. The method of claim 1 wherein determining whether the at least one sensor is compromised comprises determining that a sequence of measurements from the sensor signal fails at least one variance test by an error amount exceeding a threshold error.

10. A system comprising:
an ultrasound scanner configured to scan an interior of a target object;
one or more processors and memory storing instructions for the processors; and
a detector, implemented on the one or more processors, configured for:
providing scanned images from the ultrasound scanner as input to a machine learning software system configured to host a convolutional neural network (CNN) model;
detecting, by the CNN model, a location of at least one internal marker inside the target object using the scanned images; and
extracting identification information stored in the at least one marker.

11. The system of claim 10 wherein the at least one marker includes at least one code or material defect.

12. The system of claim 11 wherein the at least one code is an embedded bar code or an embedded quick response (QR) code.

13. The system of claim 11 wherein the at least one code is used to authenticate the material object.

14. The system of claim 10 wherein the target object comprises a three dimensional (3D) printed part.

15. The system of claim 10 wherein the target object comprises one or more of a metallic material, a plastic material, or a composite material.

16. The system of claim 10 wherein the CNN model utilizes explainable artificial intelligence (XAI).

17. The system of claim 10 wherein the ultrasound scanner comprises a transducer element.

18. The system of claim 10 wherein determining whether the at least one sensor is compromised comprises detecting if the at least one sensor is malfunctioning and/or the at least one sensor is processing an incorrect signal.

19. A non-transitory computer readable medium having stored thereon executable instructions embodied in the non-transitory computer readable medium that when executed by at least one processor of a computer cause the computer to perform steps comprising:
received scanned images from ultrasound imaging to scan an interior of a target object;
providing the scanned images generated by the ultrasound imaging as input to a machine learning software system configured to host a convolutional neural network (CNN) model;
detecting, by the CNN model, a location of at least one internal marker inside the target object using the scanned images; and
extracting identification information stored in the at least one marker.

20. The non-transitory computer readable medium of claim 19, wherein the at least one marker includes at least one code or material defect, wherein the at least one code is an embedded bar code or an embedded quick response (QR) code, and wherein the at least one code is used to authenticate the material object.
